(19) United States
(12) Patent Application Publication
(10) Pub. No.: US 2026/0083414 A1
(43) Pub. Date: Mar. 26, 2026

(54) PROTECTIVE APPARATUS FOR ARRANGEMENT ON AN OMNIDIRECTIONAL WHEEL FOR A MOBILE MEDICAL DEVICE AND MEDICAL DEVICE

(71) Applicant: Siemens Healthineers AG, Forchheim (DE)

(72) Inventors: Zsolt Pavel, Poxdorf (DE); Norbert Scherer, Pinzberg (DE)

(21) Appl. No.: 19/340,846

(22) Filed: Sep. 25, 2025

(30) Foreign Application Priority Data
Sep. 26, 2024 (DE) 10 2024 209 359.9

(51) Int. Cl. A61B 6/10; A61B 6/00

(52) U.S. Cl. CPC A61B 6/102; A61B 6/4405

(57) ABSTRACT

For collision monitoring, a protective apparatus is provided for arrangement on an omnidirectional wheel for a mobile medical device, having a substantially ring-shaped frame with a top side, a bottom side, and deflection elements fastened to the bottom side of the frame. The frame may be mounted on the omnidirectional wheel at a right angle with respect to a wheel plane, such that the frame surrounds the omnidirectional wheel in a ring-shaped manner, the bottom side is facing a surface, and the deflection elements extend along their longitudinal axis in a direction of the surface. A sensor is arranged on the bottom side of the frame and is configured to detect a deformation or deflection of one of the deflection elements by the one deflection element being brought from a first non-deformed or non-deflected position into a second deformed or deflected position.

### This application claims the benefit of German Patent Application No. DE 10 2024 209 359.9, filed on Sep. 26, 2024, which is hereby incorporated by reference in its entirety.

### BACKGROUND

The present embodiments relate to a protective apparatus for arrangement on an omnidirectional wheel for a mobile medical device and a medical device.

One major problem with automatic, motorized movement or travel of a mobile medical device (e.g., a mobile C-arm X-ray device) in an examination room or operating room is that collisions with other devices or objects may occur. To prevent this, it is known to provide a collision detection system (e.g., with proximity sensors that are arranged on the equipment cart) in order to detect obstacles before a collision occurs. In this regard, however, small objects such as cables may be easily overlooked. With such small objects, for example, there is a high risk that the wheels of the equipment cart of the medical device will strike the cable, and the cable will get under/into the wheels, for example. In such cases, the corresponding cable or wheel may be damaged, or the equipment cart may get tangled up with the cable and thus be hindered in terms of its movement. In the worst case, this may lead to serious damage to devices or endanger persons. To prevent this, an operator checks the planned path of the medical device, or this is carried out by cameras; however, this is time-consuming and may be avoided especially with self-propelled devices.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, an apparatus for a mobile medical device that is able to avoid a hazard even as a result of small obstacles, and a corresponding medical device are provided.

The protective apparatus according to the present embodiments for arrangement on an omnidirectional wheel that may move on a surface (e.g., floor of an examination room), for a mobile medical device, has a substantially ring-shaped frame with a top side, a bottom side, and a plurality of deformable or deflectable deflection elements fastened to the bottom side of the frame. The frame may be mounted on the omnidirectional wheel at a right angle to the wheel plane, such that it surrounds the omnidirectional wheel in a ring-shaped manner, the bottom side is facing the surface, and the deflection elements extend along their longitudinal axis (e.g., substantially at a right angle) in the direction of the surface. At least one sensor is arranged on the bottom side of the frame and is configured to detect a deformation or deflection of at least one of the deflection elements in that at least one deflection element may be brought from a first, non-deformed or non-deflected position into a second, deformed or deflected position (e.g., by an external force effect). In the second position, the deflection element touches the sensor (e.g., contacts the same (electrically) or exerts pressure on the sensor). The protective apparatus may be arranged on one or (e.g., in multiple versions) a number of (e.g., several) omnidirectional wheels of an equipment cart of a medical device.

Due to the ability of the deflection elements arranged on the bottom side of the frame to deform or deflect, the sensitivity of the protective apparatus is provided even in the case of small obstacles such as cables.

If the omnidirectional wheel strikes a cable (or another small obstacle) that cannot be easily pushed away, at least one or more deflection elements are deformed or deflected from a first position into a second position and pressed against the sensor. Touch (e.g., contact) or pressure causes the obstacle to be detected. As a result, automatic measures may then be initiated in order to indicate the danger (e.g., by a warning signal or a message to an operator, or to eliminate the danger, such as by stopping or reversing the movement of the wheel(s) (equipment cart)). Using the protective apparatus, collision detection may also be provided for small obstacles that are otherwise difficult to detect. The risk of damage to cables, other objects, and the medical device itself is significantly reduced by the protective apparatus. In the case of very light obstacles, the deflection elements may alternatively remove the obstacle from the path of the omnidirectional wheel and thus provide free and undisturbed locomotion.

According to one embodiment, the frame is configured to be substantially flat, and the frame plane, when mounted on the wheel, is substantially parallel to the surface on which the omnidirectional wheel may be moved. The frame may thus be easily manufactured and assembled.

According to a further embodiment, the deflection elements are elongated (e.g., cylindrical or column-shaped) and extend at a right angle to the frame plane. The deflection elements may also have an appendage (e.g., arranged laterally).

The present embodiments also include a medical device having an equipment cart with at least two (e.g., four) omnidirectional wheels, on which a protective apparatus described above is arranged. For example, the medical device has a control unit. The control unit is configured to control an action of the medical device. Such an action may be, for example, the output of a (e.g., visual or acoustic) display or an emergency stop or a reverse movement of the device.

The medical device may be configured as a mobile C-arm X-ray device. For example, the medical device has four motor-controlled omnidirectional wheels, each with a protective apparatus. The medical device is self-propelled when controlled by the control unit.

According to one embodiment, the sensor is configured as a contact sensor and/or pressure sensor. These types of sensors represent a simple and effective design of the sensor element for detecting contact by at least one of the deflection elements. A pressure sensor may, for example, be formed by a connecting block surrounded by a protective element. The connecting block is pressed by the deflection element when force/pressure is exerted and thus detects the collision. Contact sensors may also be configured based on capacitive or inductive sensors, for example, that are activated accordingly.

According to a further embodiment, the protective apparatus has a signal transmission unit that, upon detection, is configured for the transmission of a signal to a control unit of a medical device. A signal is transmitted in the event that a touch is detected by the respective sensor. The signal transmission unit may be configured, for example, for wireless signal transmission (e.g., based on radio, WLAN, Bluetooth, or other known methods).

According to a further embodiment, the sensor is configured to be ring-shaped (e.g., in a shape similar to or adapted to the frame). The sensor element may therefore also be ring-shaped, for example, and the corresponding ring on the bottom side of the frame may be arranged slightly apart from the deflection elements disposed in a first position. A large number of individual sensor elements, which together fulfil the function of the sensor, may also be provided. For example, there may be one sensor element for each deflection element. These may then be arranged along the frame, for example, so that at least one sensor element detects an obstacle (e.g., through touch or electrical contact or pressure) when a deflection element is deformed into a second position.

According to a further embodiment, the frame is configured to be closed. For example, it may easily enclose one omnidirectional wheel at a time and be arranged on it. For example, the frame is circular or rectangular in shape. The rectangular shape may have rounded corners.

According to a further embodiment, the deflection elements are arranged in a distributed manner along the frame. In this way, seamless monitoring is possible, so that no obstacle goes undetected. There are few or no deflection elements in the area of the axle, and a particularly large number of deflection elements are arranged in the area of the rolling surface or the rollers of the omnidirectional wheel, because they are always the first to encounter an obstacle. For example, the deflection elements are elongated and fastened to the frame such that the deflection elements extend at a right angle to the plane of the frame and do not directly touch the floor of the examination room.

### BRIEF DESCRIPTION OF THE DRAWINGS

FIG. 1 shows a perspective plan view of a top side of a protective apparatus;

FIG. 2 shows a perspective plan view of a bottom side of a protective apparatus;

FIG. 3 shows a top view of a protective apparatus arranged on an omnidirectional wheel;

FIG. 4 shows a side view of a protective apparatus arranged on an omnidirectional wheel;

FIG. 5 shows a deflection element in a first position;

FIG. 6 shows a deflection element in a second position; and

FIG. 7 shows a view of a mobile C-arm X-ray device with four wheels, each with a protective apparatus.

### DETAILED DESCRIPTION

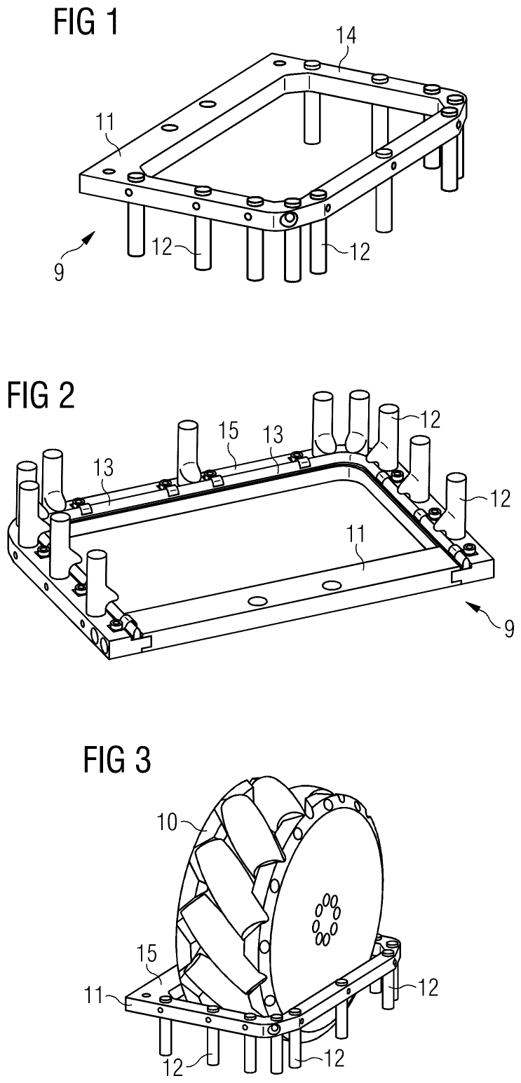

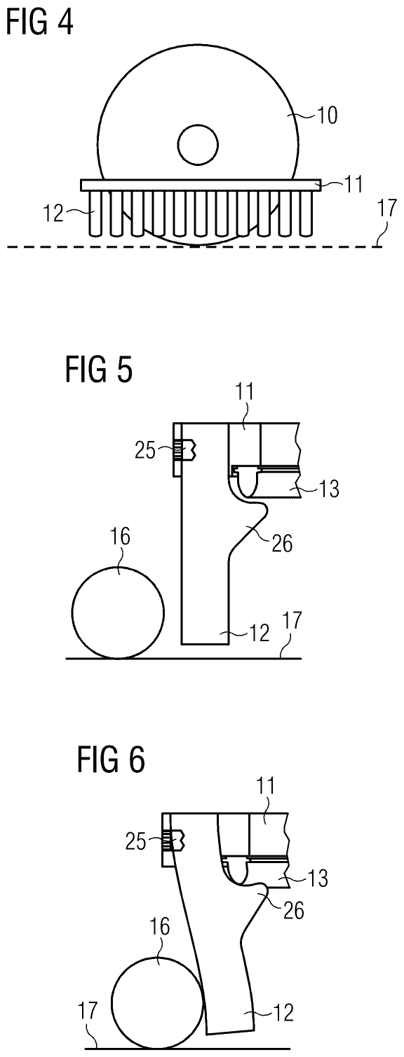

FIG. 1 shows a perspective plan view from above and FIG. 2 shows a perspective plan view from below of a protective apparatus 9 for an omnidirectional wheel of a medical device. The protective apparatus 9 has a frame 11 that has a top side 14 and a bottom side 15, with a large number of deflection elements 12 and a sensor 13 being arranged on the bottom side 15. The frame 11 is ring-shaped (e.g., in a rounded rectangular shape) and may be flat, so that the frame 11 has a frame plane. The deflection elements 12 are arranged on the bottom side 15 of the frame 11 (e.g., fastened by a fastening element 25, such as a screw, nail, clamp, etc.) or inserted into the bottom side 15 of the frame 11. The deflection elements 12 are cylindrical or columnar, for example, and extend along their longitudinal axis (e.g., substantially at a right angle to the frame plane of the frame 11). The deflection elements 12 may also be arranged at an angle to the frame plane. In the example shown, the deflection elements 12 also have an attached appendage 26 that is advantageous for improved activation of the sensor 13 and thus increases the sensitivity of the protective apparatus. When the protective apparatus 9 is mounted on an omnidirectional wheel 10, the deflection elements 12 extend in their longitudinal orientation towards the surface 17 (e.g., to the floor of the examination room), but do not touch the surface 17 (see, e.g., in FIG. 4). When mounted, the omnidirectional wheel is located in the receiving opening of the frame. For example, the frame plane is parallel to the surface 17. In one embodiment, the deflection elements 12 have a small distance to the (e.g., flat) surface (e.g., between 1 and 5 mm), so that the deflection elements 12 are not deformed or deflected by accidental contact with the floor and so that the deflection elements 12 may travel unhindered over steps and thresholds. The undeformed or undeflected basic shape of the respective deflection element 12 corresponds to a first, undeformed position, shown in FIG. 5 on a single deflection element 12. The cable 16 shown is still sufficiently far away from the deflection element 12 not to provoke any deformation or deflection.

The deflection elements 12 are distributed along the frame 11. In this case, a number of (e.g., several) deflection elements 12 are mounted in close succession along the side(s) of the frame 11, which are mounted in front of the rolling surface of the omnidirectional wheel 10 in order to provide particularly good monitoring even with very small obstacles. Fewer deflection elements 12 are arranged along the side(s) of the frame 11 that are mounted in an area of axles of the omnidirectional wheel 10. An omnidirectional wheel 10 with a protective apparatus 9 mounted on the omnidirectional wheel 10 is shown in FIG. 3. The protective apparatus 9 may be mounted on the omnidirectional wheel 10, for example, by screwing, clamping, gluing, or another fastening type to the wheel itself or the wheel axle.

In addition to the cylindrical or columnar shape, the deflection elements 12 each have an attached appendage 26. The sensor 13, which is located on the bottom side 15 of the frame 11, may also be ring-shaped, partially ring-shaped, or adapted according to the shape of the frame 11. A large number of sensor elements may also be provided. The sensor 13 is arranged relative to the deflection elements 12, such that a deformation or deflection of at least one deflection element 12 causes contact between the deflection element 12 and the sensor 13 or pressure on the sensor 13, especially in the area of the appendage. This may provide that the sensor 13 is arranged behind the deflection elements 12 or closer than the deflection elements 12 to the receiving opening of the frame provided for the wheel. The appendage 26 (if any) is arranged on the side of the respective deflection element 12 facing the sensor 13. Depending on the type of sensor 13, a signal may then be generated by touch, electrical contact, or pressure, for example. In FIG. 6, a deflection element 12 is shown in a second, deformed position, where contact with the sensor 13 occurs. This corresponds to the detection of an obstacle (e.g., in the form of the cable 16) that has collided with the deflection element 12 or is at least touching the deflection element 12.

The deflection elements 12 are configured to be flexibly deformable (e.g., made of a rubber compound or a deformable plastic). If necessary, a corresponding degree of deformability may be used for the respective protective apparatus 9. The deflection elements may, for example, have an adequate degree of stability (e.g., not too soft) to avoid unnecessary false alarms, to be able to push light obstacles out of the way by the protective apparatus, and to be able to exert the necessary pressure (e.g., in the case of a pressure sensor). At the same time, sufficient flexibility is to be provided so that in the case of more robust obstacles, the corresponding deflection element 12 is deformed into a second position by an external force (e.g., the obstacle) in order as a result to be able to detect the obstacle. In addition, the deflection elements may also be configured or arranged in a deflectable manner (e.g., by arranging a spring element as a fastening to the frame).

The sensor 13 may be configured as a touch sensor, contact sensor, and/or pressure sensor, for example. The pressure sensor may include a connecting block, for example, surrounded by a protective element. The connecting block is pressed by the deflection element when force/pressure is exerted and thus detects an obstacle. Contact sensors may also be configured based on capacitive or inductive sensors, for example, that are activated accordingly or, for example, by closing a current circuit upon contact with the deflection element.

The protective apparatus also has a signal transmission unit that is configured to forward a signal when an obstacle is detected, for example, to a control unit (e.g., a controller) of a medical device. The control unit may then control appropriate measures as a result of the forwarded signal (e.g., a movement stop or a reversal of movement of the medical device and/or an output of a visual or acoustic warning).

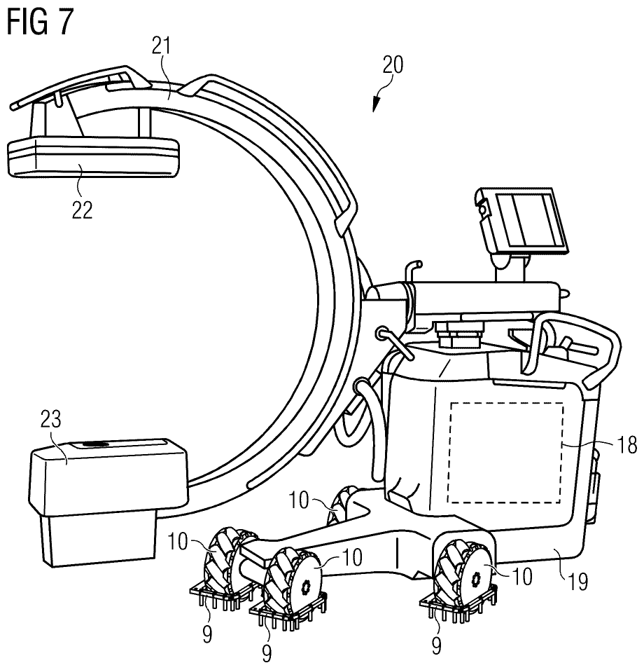

FIG. 7 shows a medical device in the form of a mobile C-arm X-ray device 20 with an equipment cart 19 with four omnidirectional wheels 10 (e.g., mecanum wheels) and protection apparatuses 9 fastened to each of the omnidirectional wheels. The equipment cart 19 carries a C-arm 21, on which an X-ray detector 22 and an X-ray source 23 are held in an adjustable manner. The mobile C-arm X-ray device 20 also has a control unit 18 that controls all actions. The mobile C-arm X-ray device 20 is self-propelled, which provides that the omnidirectional wheels 10 have drives that may be controlled by the control unit 18 to move in any direction. For example, the mobile C-arm X-ray device 20 may be moved from one examination room to another or to a desired position within an examination room. In the event that an obstacle is detected, the signal transmission units of the respective protective apparatuses 9 send a signal to the control unit 18, which then triggers the corresponding action (e.g., a movement stop or reversal of movement of the mobile C-arm X-ray device 20 or an output of a warning to a display unit 24 or by a microphone).

For assembly, the frame 11 is placed around the omnidirectional wheel 10 and fastened to the omnidirectional wheel or its axle, for example. The bottom side 15 of the frame 11 faces the surface 17 when connected to the wheel. The frame 11 has flexible deflection elements (e.g. made of rubber) that, for example, steer a light obstacle out of the way and detect a heavier obstacle. Flexibility is important so that crossing a threshold or step may be done without hindrance. To avoid the event that the wheel strikes a cable, a conductive sensor is placed directly behind the deflection elements, for example, so that, thanks to a special geometry, in the event of a collision, the sensor is activated. The sensor being activated sends a signal to the central control unit that may, for example, perform an emergency stop. For example, small obstacles such as cables may be detected, and this prevents the cables from being damaged and the medical device from being hindered in its movement.

Independent of the grammatical term usage, individuals with male, female, or other gender identities are included within the term.

The present embodiments may be summarized briefly as follows: For good collision monitoring even with small obstacles, a protective apparatus is provided for arrangement on an omnidirectional wheel for a mobile medical device. The protective apparatus includes a substantially ring-shaped frame with a top side, a bottom side, and a plurality of deformable or deflectable deflection elements fastened to the bottom side of the frame. The frame may be mounted on the omnidirectional wheel at a right angle with respect to a wheel plane, such that it surrounds the omnidirectional wheel in a ring-shaped manner, the bottom side is facing a surface, and the deflection elements extend along their longitudinal axis (e.g., substantially at a right angle) in a direction of the surface. At least one sensor is arranged on the bottom side of the frame and is configured to detect a deformation or deflection of at least one of the deflection elements, by at least one deflection element (e.g., by an external force effect) being brought from a first non-deformed or non-deflected position into a second deformed or deflected position. In the second position, the deflection element touches the sensor (e.g., makes contact therewith or exerts pressure).

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A protective apparatus for arrangement on an omnidirectional wheel that is movable on a surface, for a mobile medical device, the protective apparatus comprising:
a frame that is substantially ring-shaped, the frame having a top side, a bottom side, and a plurality of deflection elements that are deformable or deflectable and are fastened to the bottom side of the frame, the frame being mountable on the omnidirectional wheel at a right angle with respect to a wheel plane, such that the frame surrounds the omnidirectional wheel in a ring-shaped manner, wherein the bottom side of the frame is facing the surface, and the plurality of deflection elements extend along a longitudinal axis of the plurality of deflection elements; and
at least one sensor arranged on the bottom side of the frame, the at least one sensor being configured to detect a deformation or deflection of at least one deflection element of the plurality of deflection elements, by the at least one deflection element, being brought from a first non-deformed or non-deflected position into a second deformed or deflected position, the at least one deflection element touching the at least one sensor in the second deformed or deflected position.

2. The protective apparatus of claim 1, wherein the plurality of deflection elements extend along a longitudinal axis of the plurality of deflection elements substantially at a right angle in a direction of the surface.

3. The protective apparatus of claim 1, wherein the at least one sensor is configured as a touch sensor or a pressure sensor.

4. The protective apparatus of claim 1, further comprising a signal transmitter that, upon detection, is configured for transmission of a signal to a controller of a medical device.

5. The protective apparatus of claim 1, wherein the at least one sensor is configured to be ring-shaped.

6. The protective device of claim 1, wherein the frame is substantially flat, and a plane of the frame is arranged parallel to the surface.

7. The protective apparatus of claim 1, wherein the plurality of deflection elements are elongated and extend at a right angle to a plane of the plane.

8. The protective apparatus of claim 1, wherein the frame is configured to be closed.

9. The protective apparatus of claim 1, wherein the plurality of deflection elements are arranged in a distributed manner along the frame.

10. The protective apparatus of claim 1, wherein the frame is configured to be circular or rectangular.

11. A medical device comprising:
an equipment cart comprising:
at least two omnidirectional wheels, each of the at least two omnidirectional wheels being fitted with a protective apparatus,
wherein the respective omnidirectional wheel is movable on a surface, and
wherein the protective apparatus comprises:
a frame that is substantially ring-shaped, the frame having a top side, a bottom side, and a plurality of deflection elements that are deformable or deflectable and are fastened to the bottom side of the frame, the frame being mountable on the omnidirectional wheel at a right angle with respect to a wheel plane, such that the frame surrounds the omnidirectional wheel in a ring-shaped manner, wherein the bottom side of the frame is facing the surface, and the plurality of deflection elements extend along a longitudinal axis of the plurality of deflection elements; and
at least one sensor arranged on the bottom side of the frame, the at least one sensor being configured to detect a deformation or deflection of at least one deflection element of the plurality of deflection elements, by the at least one deflection element, being brought from a first non-deformed or non-deflected position into a second deformed or deflected position, the at least one deflection element touching the at least one sensor in the second deformed or deflected position.

12. The medical device of claim 11, wherein the at least two omnidirectional wheels comprise four omnidirectional wheels.

13. The medical device of claim 12, further comprising a controller configured to control an action of the medical device.

14. The medical device of claim 13, wherein the medical device is self-propelled when controlled by the controller.

15. The medical device of claim 11, wherein the medical device is configured as a mobile C-arm X-ray device.
